(19) United States
(12) Patent Application Publication
(10) Pub. No.: US 2002/0173802 A1
(43) Pub. Date: Nov. 21, 2002

(54) Conical holder for assisting vessel eversion

(76) Inventor: Dale R. Schulze, Lebanon, OH (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/861,974

(22) Filed: May 21, 2001

(51) Int. Cl. A61B 17/04

(52) U.S. Cl. 606/149

(57) ABSTRACT

An instrument and a method of use are provided for everting an end of a vessel over an end of a tubular workpiece. The instrument comprises a holder made of a sheet of spring like material rolled into a conical shape having a proximal end with a large diameter relative to the vessel diameter and a distal end with a small diameter relative to the diameter of the vessel. The method of use comprises the steps of providing the instrument, positioning the proximal end of the instrument over the end of the tubular workpiece holding the vessel, everting the end of the vessel over the proximal end of the instrument, pushing against the everted vessel and the proximal end of the instrument until the instrument is completely over the tubular workpiece and the everted vessel is radially expanded and positioned over the end of the tubular workpiece with the instrument between the vessel and the tubular workpiece, and removing the instrument from the tubular workpiece, leaving the everted end of the vessel on the end of the tubular workpiece.

### FIELD OF THE INVENTION

The field of art to which this invention relates is medical devices, more specifically, medical devices and surgical procedures for performing anastomosis of hollow organs such as blood vessels.

### BACKGROUND OF THE INVENTION

Anastomosis surgical procedures are common in the field of cardiac surgery. These procedures are conventionally used for repairing a damaged or diseased blood vessel. In a typical anastomosis procedure, a surgeon joins a first blood vessel to a second blood vessel and creates a passageway between the two blood vessels to provide for the communication of blood flow. For this kind of anastomosis, the surgeon typically uses specialized grasping tools to manipulate a tiny, curved needle attached to an extremely fine surgical filament (under 0.001 inch diameter) to suture the vessels together. The vessels may be joined end-to-end, end-to-side, or side-to-side. To facilitate healing of the joined vessels, the prevailing standard of care requires that the surgeon suture the inside surfaces of the first and second vessels together, intima to intima. The surgeon must take great care not to damage the intima of each vessel so that endothelial cells may form over the anastomosis without the formation of thrombus or other complications, thus improving the likelihood of a long term patency of the vessels. For life-saving procedures such as coronary artery bypass graft surgery (CABG), this is especially important. When performing a distal anastomosis in a conventional CABG procedure, the surgeon typically sutures an end-to-side anastomosis of a distal end of a graft vessel (such as a segment of saphenous vein harvested from the patient) to a side of a target vessel (the stenosed coronary artery). For a proximal anastomosis in a conventional CABG procedure, the surgeon sutures a proximal end of the graft vessel to the side of the aorta

As this field of art has progressed over the last several years, new anastomotic methods have been developed and introduced in attempts to replace the suturing technique briefly described above. Many of these methods incorporate novel fasteners and fastener appliers. The requirement, however, to maintain intima-to-intima contact of the joined vessels remains just as important with these approaches. In fact it is often necessary, prior to joining the vessels, for the surgeon to evert (i.e., turn inside out) the end of at least one of the vessels over the end of a member such as a tube, ferrule, or bushing, etc., which is a component of the fastener or fastener applier. This exposes the intima of that vessel for presentation to the intima of the other vessel prior to fastening the vessels.

Although it is possible to evert larger vessels (over 5 mm in diameter) using standard forceps and graspers available in the operating room, such methods are slow and may result in excessive damage to the vessel everted. And, often the surgeon requires assistance in performing the eversion procedure. Furthermore, vessels smaller than 5 mm are very difficult, if not impossible, to evert using such methods.

There are several requirements for an effective vessel eversion device. As noted earlier, for proper healing, it is important not to injure the intima of either vessel during the eversion procedure. The eversion device also must be easy for the surgeon to use without assistance and require only a few steps to operate. The eversion device must be useful for a wide range of blood vessel sizes, particularly small vessels, e.g., having a diameter of about 2-3 mm or less. In addition, it is desirable for the eversion device to be useful on one end of a vessel, when the opposite end is already attached to the patient (e.g., at the distal anastomosis of a patient undergoing a CABG procedure). The eversion device should also allow for the proper length of everted tissue, depending on the requirements of the anastomosis device or method to be used. Finally, it is desirable that the eversion device is low cost and yet operates reliably.

Accordingly, there is a need in this art for novel devices and methods for engaging and everting the end of a blood vessel (or other tubular body organ), which can be used in a quick and effective manner without causing trauma to the vessel or the intima of the vessel (or tubular body organ).

### SUMMARY OF THE INVENTION

It is an object of the present invention to provide novel eversion devices which are easy for the surgeon to use without assistance, and which efficiently and effectively engage blood vessels and evert the ends of blood vessels, including blood vessels having small or fine diameters.

A further object of the present invention is to provide novel eversion devices, which engage blood vessels and evert the ends of blood vessels without causing trauma to the blood vessel or the intima of the blood vessels.

It is yet another object of the present invention to provide novel methods of engaging and everting blood vessels quickly and efficiently, while preventing or minimizing damage to the blood vessels and the intimas of the blood vessels.

It is still yet a further object of the present invention to provide a novel vessel eversion device and procedure for everting one end of a vessel having the other end already attached to another vessel.

Accordingly, an eversion instrument for everting an end of a vessel is disclosed. The instrument has a member made of a sheet of resilient material that is formed into a conical shape. The member has a distal end with a distal opening and a proximal end with a proximal opening, and an inner passage in communication with each of said openings. The member has an outer surface.

Another aspect of the present invention is a system for everting a blood vessel. The system has an eversion instrument for everting an end of a vessel. The instrument has a member made of a sheet of resilient material that is formed into a conical shape. The member has a distal end with a distal opening and a proximal end with a proximal opening, and an inner passage in communication with each of said openings. The member has an outer surface. The system also has a tubular work piece. The tubular work piece is a tubular member having a distal end, a proximal end, an inner passage or lumen therethrough in communication with distal and proximal openings, an outer diameter, and an outer surface.

Yet another aspect of the present invention is a method of using the previously described eversion system of the present invention to evert a blood vessel. The blood vessel has a distal end, and an inner passage or lumen, and a diameter. A tubular workpiece is provided. The tubular workpiece is a tubular member having an inner passage or lumen therethrough in communication with distal and proximal openings, a proximal end, a distal end, an outer diameter, and an outer surface. An instrument is provided. The instrument is a member made of a sheet of resilient material formed into a conical shape. The member has a distal end with a distal opening and a proximal end with a proximal opening, an inner passage in communication with each of said openings, and an outer surface. The distal end has a diameter smaller than the diameter of the vessel, and a proximal end has a diameter larger than the outer diameter of the tubular workpiece. The vessel is positioned through the inner passage of the tubular workpiece such that the distal end of the vessel extends out of the distal end and distal opening of the tubular workpiece. The proximal end of the member is positioned over the tubular workpiece so that the distal end of the vessel extends from said distal end of said member. The end of the vessel is everted over said distal end of the member onto the outer surface of the member. The everted vessel and said distal end of said member are pushed simultaneously in a proximal direction until the member is completely over the workpiece and the everted vessel is radially expanded and positioned over the outer surface of the distal end of the tubular workpiece with the member between the vessel and the tubular workpiece. Then, the member is removed from said workpiece holding the everted end of the vessel.

The foregoing and other features and advantages of the present invention will become more apparent from the following description and accompanying drawings.

### BRIEF DESCRIPTION OF THE DRAWINGS

FIG. 1 illustrates the end of a blood vessel having suture filaments attached thereto.

FIG. 2 illustrates a step of threading suture filaments 80 through a cone 10 that is positioned over a tube 90.

FIG. 3 illustrates a step of pulling a vessel portion 102 of vessel 100 through cone 10.

FIG. 4 illustrates a step of inserting a mandrel 60 into vessel portion 102 and cone 10.

FIG. 5 illustrates a step of everting vessel portion 102 over cone 10.

FIG. 6 illustrates a step of removing mandrel 60 and a step of moving cone 10 and everted vessel portion 102 over tube 90.

FIG. 7 illustrates a step of removing cone 10.

FIG. 8 illustrates a step of altering the configuration of everted vessel portion 102.

FIG. 9 illustrates the use of the conical member 10 to evert the end of the blood vessel without using a tubular workpiece.

### DETAILED DESCRIPTION OF THE INVENTION

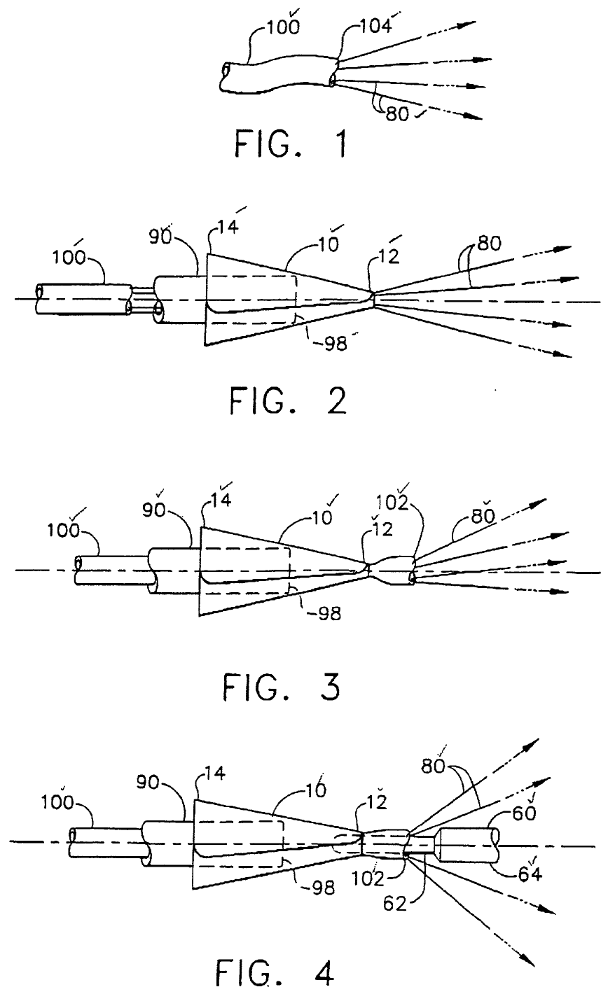

FIG. 2 is a side view of the conical holder or instrument 10 of the present invention, also referred to as a cone 10, for assisting in the eversion of a vessel 100 over the end of a tubular workpiece 90, also referred to as a tube 90. Vessel 100 may be a blood vessel, such as a segment of the greater saphenous vein, or a section of an artery, preferably for example having a diameter of about 2-8 mm, although other diameter blood vessels and other hollow organs may be everted using cone 10. Tube 90 holds vessel 100 and is representative of numerous kinds of bushings, ferrules, tubes, and specialized devices, preferably having an approximately cylindrical shape with an axial bore through it. Although it is preferred that the tube 90 have a circular cross-section, other geometric cross-sections may be used as well including oval, polygonal, combinations thereof and the like. The operator, an assistant, or a mechanical holding device holds tube 90 as the operator uses cone 10 to evert (invaginate, turn inside out) vessel portion 102 onto tube 90.

Cone 10 is seen to have a distal end 12 and a proximal end 14. When unconstrained, cone 10 normally has a conical shape with distal end 12 having a smaller diameter than the diameter of proximal end 14. Cone 10 is made from a sheet 5 of stiff but resilient material. The material has sufficient resiliency to effectively return to its original shape after being elastically deformed. The materials include, for example, any one the following: cellulose acetate propionate (CAP), polycarbonate (PC), stainless steel foil (shimstock), super elastic Nitinol, shape memory effect Nitinol, combinations thereof, equivalents thereof and the like. The thickness of the sheet 5 material will vary with the characteristics of the material and the desired resiliency of the cone 10. Cone 10 is made preferably of a transparent material such as clear PC or clear CAP for visualization of vessel 100 during the eversion method steps. One method for constructing cone 10 from PC or CAP sheet is to cut a flat piece using a pattern, roll the flat piece into a funnel shape and place in a holding fixture, place the fixture in an oven that is set at the material deflection temperature (about 280 F for PC; 192 F for CAP) for about 1-2 minutes to soften the material, and then permit the fixture and material to cool to room temperature before removing the cone from the fixture. Other conventional forming processes and equivalents thereof may be used as well. The manufacture of cone 10 from metal sheet is done using conventional metal forming processes for the metal materials.

The basic principle of the present invention is that it is easier to evert a vessel having a particular inside, unstretched diameter over an end of a rigid, circular cylindrical element having a smaller diameter rather than a bigger diameter. The method of use of cone 10, depicted in FIGS. 1-9, provides numerous advantages for the operator and the recipient of the vessel graft. Cone 10 holds and positions vessel 100 effectively during manipulation of vessel portion 102 (FIG. 3), preventing vessel portion 102 from receding into the axial bore 91 of tube 90. Cone 10 also protects vessel 100 from sharp edges or protrusions from the tube end 98, thus reducing the likelihood of injury to the vessel 100 thereby helping to ensure its long term patency once implanted in the patient. As will be next described, once the operator has invaginated end 102 of vessel 100 over distal end 12 of cone 10 while the vessel walls are relatively non-stressed, end 102 of vessel 100 is uniformly and atraumatically stretched to the minimal diameter necessary to fit over tube end 98. This reduces the risk of tearing vessel 100, again helping to ensure long-term patency of the implanted vessel. Finally, the operator may use cone 10 as a sort of staging platform on which vessel 100 is everted, positioned, and inspected for tears or other types of trauma to the intimal layers. If not satisfied with the appearance of everted vessel 100 while still positioned over cone 10, the operator may easily slip everted vessel portion 102 off of cone 10, thus un-everting vessel end 102, trim 20 vessel portion 102 with a scissors or scalpel if desired, and then repeat the steps described for FIGS. 1-6.

The method of use of cone 10 is next described in detail for FIGS. 1-8. Referring first to FIG. 1, the surgeon or assistant attaches a plurality of suture filaments 80 to a vessel end 102 of vessel 100. Suture filaments 80 may be any one of several, commercially available, conventional sutures, including but not limited to those made of polypropylene monofilament suture and polyester braided suture. Each one of suture filaments 80 typically has a needle attached to one or both ends and is passed through vessel end 102 a sufficiently effective distance from cut edge 104, preferably at least one millimeter from the cut edge. The surgeon preferably passes two spaced-apart suture filaments 80 through vessel end 104, although one suture filament or more than two suture filaments may be used.

As shown in FIG. 2, the operator next threads suture filaments 80 through tube 90 and cone 10, which is positioned over tube 90 so that tube end 98 abuts against the inside surface 15 of cone 10 approximately midway between proximal end 14 and distal end 12 of cone 10.

As shown in FIG. 3, while holding cone 10 and tube 90 together, the operator pulls vessel portion 102 through distal end 12 of cone 10. Due to the spring-like characteristic of cone 10, distal end 12 expands slightly and constricts onto vessel 100, and the bulkiness of vessel portion 102 relative to opening 13 of distal end 12 helps to prevent vessel portion 102 from receding back into interior passage 11 cone 10. The length of vessel portion 102 extending from distal end 12 of cone 10 depends on the desired length of everted vessel. The operator may pull on suture filaments 80 to lengthen vessel portion 102, or squeeze slightly on proximal end 14 of cone 10 to expand distal end 12 and reposition cone 10 on vessel 100 to shorten vessel portion 102.

FIG. 4 shows an optional step of inserting a distal end 62 of a mandrel 60 into vessel 100 and partially into cone 10. A widened portion 64 of mandrel 60 may also be inserted into vessel portion 102 to dilate vessel portion 102 prior to invagination over distal end 12 of cone 10. Mandrel 60 helps to hold vessel portion 102 in longitudinal alignment with cone 10 while the operator pulls suture filaments 80 in the proximal (left) direction to evert vessel portion 102 over distal end 12 of cone 10, as shown in FIG. 5. Mandrel 60 is especially useful for everting small vessels having a relaxed diameter of only 2-3 mm. Larger vessels (greater than 3 mm diameter) may usually be everted without the use of mandrel 60. When not using optional mandrel 60, the surgeon pulls the sutures 80 proximally to evert vessel end 102 over distal cone end 12.

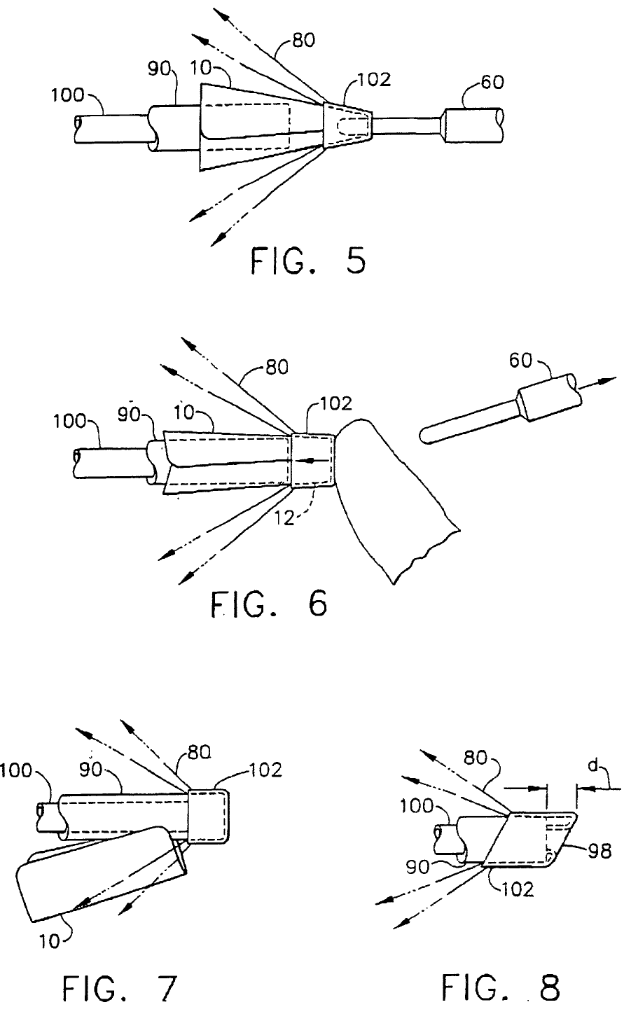

The operator next removes mandrel 60 from vessel 100 if mandrel 60 was used to facilitate eversion of vessel portion 102 onto cone 10. As shown in FIG. 6, the operator pushes distal end 12 of cone 10 with a fingertip or an available surgical instrument such as a forceps handle, to transfer everted vessel portion 102 over tube 90. As the operator pushes, distal end 12 of cone 10 and everted vessel portion 102 expand radially to fit over tube 90. During this step, the operator or an assistant may also pull suture filaments 80 in the proximal (left) direction to facilitate the positioning of everted vessel portion 102 over tube 90. Cone 10 changes from a funnel shape to an approximately tubular shape during this step.

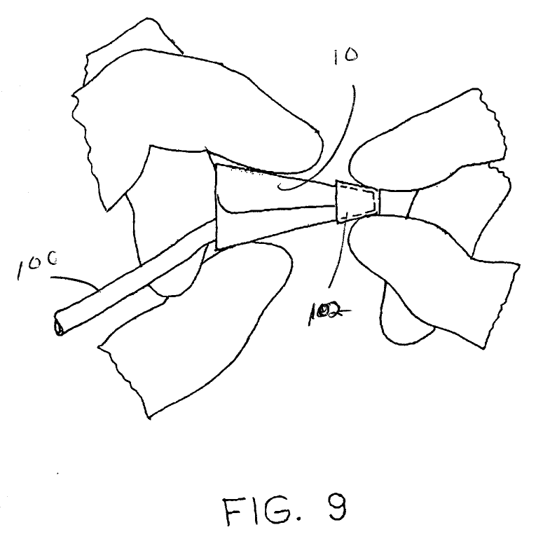

As shown in FIG. 7, the operator next removes cone 10 from between tube 90 and everted vessel portion 102 by partially unraveling sheet 5 so that the ends are spread apart, thereby completing the method of use of cone 10. As noted earlier, prior to removing cone 10, the eversion of vessel portion 102 may be inspected, and even reversed if desired, in order to repeat the steps described for FIGS. 1-6. Once removed, cone 10 resumes a funnel shape and may be reused for another vessel eversion if desired. As shown in FIG. 8, the operator may use suture filaments 80 to alter the configuration of everted vessel portion 102, either while cone 10 is removed or still attached to tube 90. The operator may create an offset distance “d” as shown in FIG. 8, for example, to incorporate a desired anastomotic method or device.

In an alternate method for using cone 10, especially for when vessel 100 is a thin-walled vein that is larger than 3 mm (arterial walls are generally thicker than venous walls), suture filaments 80 are not used. Instead, the operator may insert vessel 100 through tube 90 and cone 10 by using any one of a number of techniques. For example, the operator may use an available surgical instrument such as a forceps to grasp the end of the vessel and pass it through tube 90 and cone 10, perhaps partially unraveling/expanding cone 10 in the process. Or the operator may devise and use a hooking probe, wire, or snare to pull vessel 100 through tube 90 and cone 10.

Cone 10 as described for the specific embodiment shown in FIGS. 2-7 is easily constructed from a relatively low cost plastic or metal and may be supplied to the end user as a sterilized unit intended for single patient use. A re-sterilizable embodiment of cone 10, intended for multi-patient use, however, will become apparent to those skilled in the art. Also, cone 10 may be made available in a variety of sizes to accommodate a wide range of sizes and types of bodily hollow organs, adaptable to eversion onto various kinds of tubular workpieces. The operator may also use cone 10 to evert a vessel onto itself, and is not limited to use for everting a vessel portion onto a tubular workpiece as seen in FIG. 9. The operator inserts vessel 100 through cone 10 so that a vessel portion 102 extends distal to distal tip 12 of cone 10, everts vessel portion 102 onto distal end 12 of cone 10, and then removes cone 10 from around vessel 100.

The advantages of the vessel eversion device and method of the present invention are numerous and include the following: holding and positioning the blood vessel during manipulation, protecting the vessel from sharp edges or protrusions from the tube end, uniformly and atraumatically stretching the vessel during the eversion method, and allowing the use of the device as a staging platform on which the vessel is everted, positioned, and inspected for tears or other types of trauma to the intimal layers.

Although this invention has been shown and described with respect to detailed embodiments thereof, it will be understood by those skilled in the art that various changes in form and detail thereof may be made without departing from the spirit and scope of the claimed invention

What is claimed is:

1. An instrument for everting an end of a vessel, said instrument, comprising:
a member made of a sheet of resilient material formed into a conical shape, said member having a distal end with a distal opening and a proximal end with a proximal opening, an inner passage in communication with each of said openings, an inner surface and an outer surface.

2. The instrument of claim 1, wherein said member is transparent.

3. The instrument of claim 1, wherein the diameter of the distal end is smaller than the diameter of a vessel and the diameter of the proximal end is larger than said distal end and larger than the diameter of a tubular workpiece, and said distal end is radially expandable to fit over said tubular workpiece, and said proximal end is radially contractible.

4. A method of everting an end of a vessel, said method comprising the steps of
providing a body vessel having a distal end, and an inner passage, said body vessel having a diameter;
providing a tubular workpiece having an inner passage, a proximal end, a distal end and an outer surface;
providing an instrument comprising a member made of a sheet of resilient material formed into a conical shape, said member having a distal end with a distal opening and a proximal end with a proximal opening, an inner passage in communication with each of said openings, an inner surface and an outer surface, the distal end having a diameter smaller than the diameter of the body vessel, and a proximal end having a diameter larger than the diameter of said tubular workpiece;
positioning the body vessel through the inner passage of the tubular workpiece such that a distal end of the body vessel extends out of the distal end of the tubular workpiece;
positioning said proximal end of said member over the tubular workpiece so that the distal end of the body vessel extends from said distal end of said member;
everting the end of the body vessel over said distal end of the member onto the outer surface of the member;
pushing simultaneously against the everted body vessel and said distal end of said member in a proximal direction until said member is completely over said workpiece and the everted body vessel is radially expanded and positioned over outer surface of the distal end of the tubular workpiece with the member between the body vessel and the outer surface of the tubular workpiece; and
removing said member from said workpiece holding said everted body vessel.

5. The method of claim 4, wherein the body vessel comprises a blood vessel.

6. The method of claim 4 wherein the body vessel additionally comprises a proximal end.

7. A system for everting a blood vessel, comprising:
I. an instrument for everting an end of a vessel, said instrument, comprising:
a member made of a sheet of reslient material formed into a conical shape, said member having a distal end with a distal opening and a proximal end with a proximal opening, an inner passage in communication with each of said openings, and an outer surface and an inner surface, said distal opening being radially expandable and said proximal opening being radially contractible; and,
II. a tubular workpiece comprising a tubular member having an inner passage, a proximal end, a distal end, a proximal opening, and a distal opening, and an outer surface, wherein said inner passage is in communication with said proximal and distal openings.
